(19) United States
(12) Patent Application Publication
(10) Pub. No.: US 2020/0093714 A1
(43) Pub. Date: Mar. 26, 2020

(54) MICROCAPSULE SYSTEM FOR POLYSENSORY OLFACTORY EFFECTS II

(71) Applicant: Henkel AG & Co. KGaA, Duesseldorf (DE)

(72) Inventors: Andreas BAUER, Kaarst (DE); Andre HAETZELT, Eimeldingen (DE); Frank PESSEL, Duesseldorf (DE); Andreas GERIGK, Erkelenz (DE); Anneliese WILSCH-IRRGANG, Duesseldorf (DE); Klaus LAST, Braunschweig (DE); Raul AMADO, Minden (DE)

(21) Appl. No.: 16/616,563

(22) PCT Filed: May 18, 2018
(86) PCT No.: PCT/EP2018/063161

§ 371 Date: Nov. 25, 2019

(30) Foreign Application Priority Data
May 24, 2017 (DE) 10 2017 111 445.9

(51) Int. Cl. A61K 8/11; A61K 8/81; A61K 8/84; A61Q 13/00; C11D 3/50

(52) U.S. Cl. CPC A61K 8/11; A61K 8/8158; A61K 8/84; A61K 2800/56; C11D 3/505; A61K 2800/30; A61K 2800/412; A61Q 13/00

(57) ABSTRACT

A microcapsule system may include an outer microcapsule having an outer capsule shell where the outer microcapsule comprises at least one inner microcapsule with an inner capsule shell and a first fragrance composition. The inner microcapsule may include a second fragrance composition that differs from the first fragrance composition. Methods for the production thereof, products that contain said microcapsules, methods for producing polysensory olfactory impressions, and to the corresponding use of the microcapsule system, are also disclosed.

### CROSS-REFERENCE TO RELATED APPLICATION

The present application is a national stage entry according to 35 U.S.C. § 371 of PCT application No.: PCT/EP2018/063141 filed on May 18, 2018; which claims priority to German Patent Application Serial No.: 10 2017 111 445.9, which was filed on May 24, 2017; both of which are incorporated herein by reference in their entirety and for all purposes.

### TECHNICAL FIELD

The invention relates to the field of perfume-containing microcapsules, as well as cosmetic agents, cleaning agents and textile treatment agents which contain perfume-containing microcapsules, and to methods for releasing fragrances from these microcapsules when using said agents.

### BACKGROUND

A large number of cosmetic agents, cleaning agents and textile treatment agents contain sensitive ingredients, such as odorants, essential oils, perfume oils and nourishing oils, dyes or antibacterial active ingredients. The disadvantage is that such ingredients that are used in agents of this kind often lose their activity during storage and/or before the desired application time, or are at least greatly reduced for example by chemical reactions due to interaction with other components of the respective agents and/or by physical influences.

In order to use substances of this kind in a controlled manner at the desired site with maximum effect, active substances such as fragrances, nourishing oils, antibacterial active ingredients and the like are often added to the products in a spatially delimited, protected form. Sensitive substances are frequently enclosed in capsules of various sizes, adsorbed on suitable carrier materials, or chemically modified. These substances can then be released by means of a suitable mechanism, for example mechanically by shearing, or directly from the matrix material by means of diffusion.

There are already numerous commercial encapsulation systems based on natural or synthetic polymers. They may enclose an active ingredient or the solution thereof and then be physically or chemically cross-linked in the shell or precipitated by a coacervation process with another polymer. Microcapsules are known from the prior art which may contain liquid, solid or gaseous substances as core material. Phenol-formaldehyde polymers, melamine-formaldehyde polymers, polyurethane, gelatin, polyamides or polyureas can be used as the material for the capsule walls, for example. Cosmetic agents, cleaning agents and textile treatment agents which contain microcapsules are known per se. In particular, microcapsules consisting of melamine-formaldehyde resins have proven to be suitable in these agents, since they are particularly stable.

For example, the European laid-open patent application EP 0 967 007 A2 describes a method for microencapsulating solid, biologically active substances, in particular pesticides, by polycondensation of a melamine-formaldehyde or phenol-formaldehyde resin or a urea-formalin resin in a dispersion in the presence of the corresponding active substance to be encapsulated and a non-ionic polymeric protective colloid for stabilizing the emulsion, with microcapsules having average particle diameters of from 0.1 to 300 μm being obtained. This method is only suitable for encapsulating solid biological active substances. In order to stabilize the emulsion, a polymeric protective colloid has to be added to the emulsion. Conventional capsule systems with a simple shell structure are described.

K. Hong “Melamine resin microcapsules containing fragrant oil: synthesis and characterization” in Materials Chemistry and Physics 58 (1999), pages 128-131, describes preparing long-life active-ingredient-containing melamine resin microcapsules containing a fragrance oil by in-situ polymerization of migrin oil as the capsule core material, melamine and formalin as the capsule shell material, sodium lauryl sulfate as an emulsifier and polyvinyl alcohol as a protective colloid. This results in capsule systems loaded with fragrance oil and having a simple shell structure.

However, the known capsule systems do not allow different fragrance profiles to be produced over the entire application cycle of a product. This may be desirable or advantageous in particular if the fragrance impression is intended to change for the consumer over time. What is conceivable above all is a first fragrance impression which is characteristic of the product or the intended use thereof and optionally also achieves a particular recognition value, for example a predominantly cosmetic odor when opening or applying the product which, after use, is replaced by a different odor, for example a predominantly fruity odor. Thus, for example, it would be possible to combine the odor of a washing and cleaning agent product which is typically primarily intended to impart freshness and cleanliness, with more complex perfumes which are only released at a later point in time after application.

International laid-open patent application WO 2012/032145 A1 describes a formaldehyde-free capsule system based on aromatic alcohols and aldehydic compounds, in which phloroglucinol/resorcinol-based capsules demonstrate the best performance, in particular with regard to boosting properties, compared with alternative technologies. Nevertheless, the disadvantage of this technology is that the resorcinol and phloroglucinol-based capsules discolor the product formulation and are therefore not commercially viable. The capsules also exhibit excessive, unacceptable sedimentation after being stored.

### SUMMARY

The object of the present invention was therefore to provide the consumer with an improved fragrance experience over the entire application cycle of a product. Such an improvement in the perception of odors can be achieved by switching odor profiles during the application of a product and/or the use of a surface provided with the product, for example a textile.

It has now surprisingly been found that the problem can be solved by the use of capsules which contain additional smaller, fragrance-loaded capsules based on aromatic alcohols and aldehydic compounds (“capsule-in-capsule systems”) in conjunction with appropriately tailored fragrance compositions.

In a first aspect, a microcapsule system comprising an outer microcapsule having an outer capsule shell is described, wherein the outer microcapsule contains:
(a) at least one inner microcapsule enclosed therein having an inner capsule shell; and
(b) a first fragrance composition;
   wherein the capsule shell of the outer microcapsule completely surrounds the inner microcapsule and the first fragrance composition, characterized in that the inner microcapsule contains a second fragrance composition which is completely surrounded by the inner capsule shell of the inner microcapsule and differs from the first fragrance composition, the capsule shell of the inner microcapsule comprising a resin which can be obtained by reacting
i. at least one aromatic alcohol or the ether or derivatives thereof with
ii. at least one aldehydic component that comprises at least two C atoms per molecule, and
iii. optionally in the presence of at least one (meth)acrylate polymer.

In another aspect, methods for producing the microcapsule systems are described herein, which comprise (1) providing microcapsules containing the second fragrance composition, and a first fragrance composition, (2) encapsulating the microcapsules containing the second fragrance composition, and the first fragrance composition, in an outer microcapsule.

In yet another aspect, agents for washing, cleaning, conditioning, caring for and/or dyeing hard or soft surfaces, for example textiles, dishes etc., which agents contain the microcapsule system are described herein.

The method additionally relates to producing polysensory fragrance impressions using the microcapsule systems described herein, wherein the first fragrance composition is firstly released from the outer microcapsule and, following a time delay, the second fragrance composition is then released from the inner microcapsule. In non-limitinq embodiments, the first fragrance composition is released, inter alia, by means of diffusion through the capsule wall of the outer microcapsule and optionally additionally by means of mechanical stress. In non-limitinq embodiments, the second fragrance composition is released by means of mechanical force, in particular by means of friction. In these methods, the microcapsule system is applied to the surface by means of contact of an agent for washing, cleaning, conditioning, caring for and/or dyeing hard or soft surfaces which contains the microcapsule system described herein, and the fragrances are then released, by means of diffusion and then mechanical force, such as by friction.

In yet another aspect, the use of the microcapsule system to produce polysensory fragrance impressions is also described.

Capsule-in-capsule systems are generally known from the international patent application WO 02/060573 A2, for example. This document describes the encapsulation of a wide range of active substances in capsule-in-capsule systems, which can respond to more than one change in environmental properties and provide good to increased protection for the encapsulated ingredients. The systems described are suitable in particular for applications in washing and cleaning agents, cosmetics and body care agents and in adhesive technology. Nevertheless, said document does not describe capsule-in-capsule systems that are intended for encapsulating at least two different fragrance compositions and that allow sequential release in order to produce polysensory fragrance impressions.

These and other aspects, features and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples.

### DETAILED DESCRIPTION

Unless indicated otherwise, all percentages indicated are percent by weight and relate to the composition/capsule/capsule shell mentioned in each case. Numerical ranges that are indicated in the format “from x to y” also include the stated values. If several preferred numerical ranges are indicated in this format, it is self-evident that all ranges that result from the combination of the various endpoints are also included.

“At least one”, as used herein, refers to one or more, for example 1, 2, 3, 4, 5, 6, 7, 8, 9 or more. In particular, this expression refers to the type of agent/compound and not to the absolute number of molecules. “At least one fragrance”, therefore, means that at least one type of fragrance is included but also that two or more different types of fragrances may be contained.

“About” or “approximately”, as used herein in connection with a numerical value, refers to the numerical value±10%, such as ±5%.

“Microcapsule system” as used herein refers to the capsule-in-capsule systems described herein, i.e. microcapsules which in turn enclose microcapsules.

“Microcapsule” as used herein refers to capsules which have a core-shell morphology on a micro-meter scale and have a capsule shell which completely encloses a core. “Completely enclosing” or “completely surrounding” as used herein in reference to the microcapsules means that the core is completely surrounded by the shell, i.e. in particular, is not embedded in a matrix so as to be exposed at one point. The capsule shell may be designed such that the release of the contents is controlled, i.e. the contents are not released in a spontaneous and uncontrolled manner independently of a release stimulus. For this reason, the capsule shell is substantially impermeable to the encapsulated contents. “Substantially impermeable” as used in this context means that the contents of the capsule or individual ingredients cannot spontaneously penetrate the shell; instead, the release can only occur by opening the capsule or, optionally, also by means of a diffusion process which takes place over a long period of time. The core may be solid, liquid and/or gaseous, but is solid and/or liquid. The microcapsules are substantially spherical and have diameters in the range of from 0.01 to 1000 μm, in particular from 0.1 to 500 μm. The capsule shell and the capsule core consist of different materials; in particular, the capsule shell is solid under standard conditions (20° C., 1013 mbar), and the core is solid and/or liquid, in particular liquid.

When referring generally to “microcapsules” below, it is clear that the corresponding statements apply to both the outer and inner microcapsules, unless it is explicitly stated that the statement applies to one of the two types of microcapsules used. Furthermore, even when the capsule system is described herein with reference to one outer microcapsule in each case, it is clear that the microcapsule system used usually contains a large number of microcapsules of this kind, typically >100, such as >1000, or substantially consists thereof (i.e. by 20 wt. % or more, such as 30 wt. % or more, i.e. 50 wt. % or more) or completely consists thereof (i.e. by 100 wt. %). In addition to the microcapsules, the microcapsule system may also comprise a liquid carrier medium, for example an aqueous carrier medium, in which the outer microcapsules are dispersed in order to form, for example, a capsule slurry. The microcapsules typically constitute 10 to 80 wt. %, such as 20 to 50 wt. %, in capsule slurries of this kind.

High-molecular compounds of animal or plant origin, for example protein compounds (gelatin, albumin, casein), cellulose derivatives (methylcellulose, ethylcellulose, cellulose acetate, cellulose nitrate, carboxymethylcellulose) and in particular synthetic polymers (e.g. polyamides, polyolefins, polyesters, polyurethanes, epoxy resins, silicone resins and condensation products of carbonyl and NH group-containing compounds) can be used very generally as the capsule material for the outer microcapsules, for example. Specifically, the shell material may be selected, for example, from polyacrylates; polyethylene; polyamides; polystyrenes; polyisoprenes; polycarbonates; polyesters; polyureas; polyurethanes; polyolefins; polysaccharides; epoxy resins; vinyl polymers; urea cross-linked with formaldehyde or glutaraldehyde; melamine cross-linked with formaldehyde; gelatin-polyphosphate coacervates, optionally cross-linked with glutaraldehyde; gelatin gum arabic coacervates; silicone resins; polyamines reacted with polyisocyanates; acrylate monomers polymerized by means of free radical polymerization; silk; wool; gelatin; cellulose; proteins; and mixtures and copolymers of the above.

In order to produce the outer microcapsules, the known microencapsulation methods are suitable in principle, in which methods, for example, the encapsulation of the phase to be encapsulated is carried out by means of coating with film-forming polymers (such as those mentioned above) which, after emulsification and coacervation or interfacial polymerization, precipitate on the material to be coated. The phase to be encapsulated is a fragrance composition, usually in the form of a perfume oil. In order to produce the outer microcapsules, the inner microcapsules are dispersed in the first fragrance composition, usually, again, a perfume oil which differs from that in the inner capsules, and this dispersion is then encapsulated in film-forming polymers.

The microcapsules may release the fragrance compositions contained by means of various environmental influences, such as when the pH or ionic strength of the environment changes, when the temperature changes, upon exposure to light, by means of diffusion and/or under mechanical stress. The outer microcapsule, i.e. the capsule shell of the outer microcapsule, and the inner microcapsule, i.e. the capsule shell of the inner microcapsule, may differ in their structure or composition, such that different release mechanisms are used or, if the same release mechanism is used, different release conditions are set. “Different release conditions” as used herein also refers to different permeabilities of the capsule shells, as already defined above. In various embodiments, the outer and inner microcapsules may differ in their release behavior, i.e. the release of the encapsulated material, for example the fragrance compositions. Such a difference in the release behavior relates to the capsules per se in direct comparison, i.e. in a comparison in which the inner capsules in free, unencapsulated form are compared with the outer capsules per se, i.e. without inner capsules encapsulated therein, in terms of release behavior. In various embodiments, inner and outer capsules differ in their release behavior when a given release mechanism, such as diffusivity, is examined. Such an examination may be carried out, for example, by means of thermogravimetric analysis (TGA) at a heating rate of 1K/min coupled with fast Fourier infrared spectroscopy (FFIR) in a temperature range of from room temperature (20° C.) to 350° C. in a nitrogen atmosphere (for example, 1.8 L N₂/h). The capsules are weighed out in the range of from 10-12 mg and examined in Al crucibles. Both types of capsules are filled with the same fragrance mixture for the purpose of this comparative test. Before each measurement, a background measurement is carried out, and the signals of the measurement spectrum are always immediately adjusted for the background. When reference is made herein to inner and outer capsules differing in their release behavior, this means, unless stated otherwise, that the capsules differ in their permeability to the encapsulated substances such that, during the dynamic phase (i.e. during heating), the TGA-FFIR results in a difference in weight loss relative to the starting weight of at least 1% under the above conditions at any point in time, for example in the temperature range between 80 and 300° C., at the same temperature. In various embodiments, the weight loss of the outer capsules under the same conditions at the same temperature (for example, after 250 minutes of heating at 1K/min and a temperature of 280° C.) is at least 1 wt. % greater than that of the inner capsules. This means, for example, that, after a period of 250 minutes and at a temperature of 280° C., the weight loss (relative to the initial weight) of the outer capsule is 86.6%, while that of the inner capsule is 82.3% (i.e. a difference of 4.3%).

The first fragrance composition may be released from the outer microcapsules by means of diffusion and, optionally, also by means of mechanical release, and for the second fragrance composition to be released from the inner microcapsules by means of a different release mechanism, in particular only by means of mechanical stress. For this purpose, it is necessary for the capsule materials for outer and inner capsule shells to accordingly be differently designed. In such embodiments, therefore, the outer and inner microcapsules differ in their release behavior. The outer microcapsules may slowly release the first fragrance composition, for example by the capsule shell being diffusively permeable, whereas the inner microcapsules are retained and are only broken up in response to a later stimulus. Even if the outer microcapsules are diffusively permeable, the inner microcapsules are not usually released by means of diffusion, but by the outer microcapsule being broken up by means of one of the other release mechanisms mentioned above. In this case, the difference in the diffusivity can also be caused by the inner capsules themselves being encapsulated, and therefore the diffusivity is limited by the encapsulation in the outer capsules, even with the same structure of the shell. In various embodiments, this difference is further reinforced by the fact that the capsule shell of the inner capsules is different from that of the outer capsules, i.e. there is a difference in release behavior, as described above. Thus, when diffusion is mentioned as the release mechanism, this always relates to the fragrances and not the inner microcapsules. “Diffusively permeable” with respect to the outer capsules means here that the permeability to the fragrance molecules by means of diffusion is greater than the corresponding permeability of the inner capsules. The term is therefore used herein substantially as a relative term.

The diffusivity (permeability to diffusion) of the capsules can be adjusted for example by the degree of cross-linking of the shell materials and the wall thickness of the capsules.

The microcapsules may be water-soluble and/or water-insoluble microcapsules.

However, in particular the outer microcapsules are water-insoluble microcapsules. The water insolubility of the outer microcapsules has the advantage that, by using appropriate washing or cleaning agents, it is possible to keep fragrances separated after application, and also has the advantage that the fragrance can be released/continue to be released from the microcapsules even after application.

The inner microcapsules are also water-insoluble for the reasons mentioned above.

The wall material of the outer microcapsules comprises polyacrylates, polyurethanes, polyolefins, polyamides, polyesters, polysaccharides, epoxy resins, silicone resins and/or polycondensation products consisting of carbonyl compounds and compounds containing NH groups. Melamine-urea-formaldehyde microcapsules or melamine-formaldehyde microcapsules or urea-formaldehyde microcapsules are non-limitinq examples. In non-limitinq embodiments, therefore, the outer microcapsules are those based on melamine-formaldehyde resins.

The general procedure in the production of microcapsules per se has long been known to a person skilled in the art. Particularly suitable methods for producing microcapsules are described in principle, e.g. in U.S. Pat. No. 3,516,941, in U.S. Pat. No. 3,415,758 or also in EP 0 026 914 A1. The latter describes, for example, producing microcapsules by means of acid-induced condensation of melamine-formaldehyde precondensates and/or their C1-C4-alkyl ethers in water, in which the hydrophobic material forming the capsule core is dispersed, in the presence of a protective colloid.

The outer microcapsules are those based on melamine-formaldehyde resins, and the encapsulated fragrances are released at least in part diffusively. In such embodiments, the capsule shell for the encapsulated fragrance composition or components thereof is permeable such that the odorant molecules diffuse, in a long-lasting manner, into the ambient air. In addition, the outer microcapsule can be broken up, in particular is friable, by means of mechanical stress. The latter mechanism causes the outer capsule shell to break up and to thus release the inner microcapsules.

As already described above, the capsule wall of the inner microcapsule comprises a resin which can be obtained by reacting
i. at least one aromatic alcohol or the ether or derivatives thereof with
ii. at least one aldehydic component that comprises at least two C atoms per molecule, and
iii. optionally in the presence of at least one
   (meth)acrylate polymer. The capsule wall of the inner microcapsule may consist substantially, i.e. by at least 50, such as at least 75, i.e. at least 90 wt. %, or entirely of one or more resins of this kind.

Aryloxyalkanols, arylalkanols and oligoalkanol aryl ethers may be used as aromatic alcohols. Aromatic compounds in which at least one free hydroxyl group, such as at least two free hydroxyl groups, are directly aromatically bonded may be used, e.g. for at least two free hydroxyl groups to be bonded directly to an aromatic ring and optionally arranged in the meta position with respect to one another. The aromatic alcohols may be selected from phenols, o-cresol, m-cresol, p-cresol, α-naphthol, μ-naphthol, thymol, pyrocatechol, resorcinol, hydroquinone, 1,4-naphthohydroquinone, phloroglucinol, pyrogallol, hydroxyhydroquinone, and mixtures thereof.

Aromatic alcohols are also those which are used in the production of polycarbonate plastics materials and epoxy resin lacquers, in particular 2,2-bis-(4-hydroxyphenyl)propane (bisphenol A). The aromatic alcohol may be selected from phenols having two or more hydroxyl groups, such as from pyrocatechol, resorcinol, hydroquinone and 1,4-naphthohydroquinone, phloroglucinol, pyrogallol, hydroxyhydroquinone and mixtures thereof, with in particular resorcinol (1,3-dihydroxybenzene) and/or phloroglucinol (1,3,5-trihydroxybenzene), and phloroglucinol.

In a further embodiment, the aromatic alcohol a) is used as an ether in the production of the inner microcapsules, the ether, in a non-limitinq embodiment, being a derivative of the relevant free form of the aromatic alcohol a) to be reacted. The free alcohol can also be present, thus resulting in a mixture. In this case, the molar ratio between the free form of the aromatic alcohol to be reacted and the additional component mentioned above (ether form of an aromatic alcohol) may be between 0:100, such as 1:1, or 1:2 or 1:4.

The advantage of mixing the aromatic alcohol with an ether form is that it is thus possible to influence the reactivity of the system. In particular, with the suitable choice of ratio, a system can be created of which the reactivity is in a balanced ratio to the storage stability of the system. As derivatives of the aromatic alcohols, the esters thereof may be used. The term “derivative” as used herein therefore includes the esters of said alcohols.

Different embodiments may use different inner microcapsules which may then differ from one another in the reacted component a).

Particularly stable inner microcapsules are obtained with the aromatic alcohols a) phloroglucinol and/or resorcinol. It is also possible to use mixtures of inner microcapsules, in each of which either phloroglucinol or resorcinol is used as component a).

Both aliphatic and aromatic aldehydes may be used as aldehydes b) having at least two C atoms. Non-limitinq aldehydes are one or more aldehydes selected from the following group of valeraldehyde, capronaldehyde, caprylaldehyde, decanal, succinic dialdehyde, cyclohexanecarbaldehyde, cyclopentanecarbaldehyde, 2-methyl-1-propanal, 2-methylpropionaldehyde, acetaldehyde, acrolein, aldosterone, antimycin A, 8′-apo-μ-caroten-8′-al, benzaldehyde, butanal, chloral, citral, citronellal, crotonaldehyde, dimethylaminobenzaldehyde, folinic acid, fosmidomycin, furfural, glutaraldehyde, glutardialdehyde, glyceraldehyde, glycolaldehyde, glyoxal, glyoxylic acid, heptanal, 2-hydroxybenzaldehyde, 3-hydroxybutanal, hydroxymethylfurfural, 4-hydroxynonenal, isobutanal, isobutyraldehyde, methacrolein, 2-methylundecanal, mucochloric acid, N-methylformamide, 2-nitrobenzaldehyde, nonanal, octanal, oleocanthal, orlistat, pentanal, phenylethanal, phycocyanin, piperonal, propanal, propenal, protocatechualdehyde, retinal, salicylaldehyde, secologanin, streptomycin, strophanthidin, tylosin, vanillin, cinnamaldehyde and mixtures thereof. Particularly stable microcapsules were obtained using the aldehydic components b) glutardialdehyde and/or succinic dialdehyde.

The aldehydic component may have at least one or two, such as two, three or four, in particular two, free aldehyde groups per molecule. The dialdehydes derived from linear C₂₋₈ alkanes, such as glyoxal, glutaric and/or succinic dialdehyde, as aldehydic components, such as glutardialdehyde.

In the inner microcapsules which can be used, the molar ratio of a), the at least one aromatic alcohol (or ether or derivative thereof), to b), the at least one aldehydic component, can generally be between 1:1 and 1:5, such as between 1-2 and 1-3, and or approximately between 1-2.6 in the case of resorcinol/phloroglucinol. The weight ratio of components a)+b) to c), i.e. the ratio of the total weight of a)+b)) to the weight of component c) is generally between 1:1 and 1:0.01, such as between 1:0.2 and 1:0.05.

If different inner microcapsules are used, they may differ from one another in a particular embodiment in terms of the reacted components b).

The optionally used (meth)acrylate polymers may be homopolymers or copolymers of methacrylate monomers and/or acrylate monomers. The term “(meth)acrylate” means both methacrylates and acrylates. The (meth)acrylate polymers are, for example, homopolymers or copolymers, such as copolymers, of one or more polar-functionalized (meth)acrylate monomers, such as (meth)acrylate monomers containing sulfonic acid groups, carboxylic acid groups, phosphoric acid groups, nitrile groups, phosphonic acid groups, ammonium groups, amine groups or nitrate groups. The polar groups can also be present in salt form. The (meth)acrylate polymers are suitable as protective colloids and can be advantageously used in the production of microcapsules.

(Meth)acrylate copolymers may consist, for example, of two or more (meth)acrylate monomers (e.g. acrylate+2-acrylamido-2-methylpropanesulfonic acid) or of one or more (meth)acrylate monomers and one or more monomers that are different from (meth)acrylate monomers (e.g. methacrylate+styrene).

Examples of (meth)acrylate polymers are homopolymers of (meth)acrylates containing sulfonic acid groups (e.g. 2-acrylamido-2-methylpropanesulfonic acid or salts thereof (AMPS), commercially available as Lupasol®PA 140, BASF), or copolymers thereof, copolymers of acrylamide and (meth)acrylic acid, copolymers of alkyl(meth)acrylates and N-vinylpyrrolidone (commercially available as Luviskol®K15, K30 or K90, BASF), copolymers of (meth)acrylates with polycarboxylates or polystyrene sulfonates, copolymers of (meth)acrylates with vinyl ethers and/or maleic anhydride, copolymers of (meth)acrylates with ethylene and/or maleic anhydride, copolymers of (meth)acrylates with isobutylene and/or maleic anhydride, or copolymers of (meth)acrylates with styrene-maleic anhydride.

Non-limiting (meth)acrylate polymers are homopolymers or copolymers, such as 2-acrylamido-2-methylpropanesulfonic acid or the salts thereof (AMPS). Non-limiting 2-acrylamido-2-methylpropanesulfonic acid or the salts thereof, e.g. copolymers having one or more comonomers from the group of (meth)acrylates, may include vinyl compounds such as vinyl esters or styrenes, unsaturated dicarboxylic or polycarboxylic acids such as maleic acid esters, or the salts of amyl compounds or allyl compounds. Hereinafter, non-limitinq comonomers for AMPS are mentioned, but these comonomers can also be copolymerized with other polar-functionalized (meth)acrylate monomers:

1) Vinyl compounds, e.g. vinyl esters such as vinyl acetate, vinyl laurate, vinyl propionate or vinyl esters of neononanoic acid, or aromatic vinyl compounds such as styrene comonomers, for example styrene, alpha-methylstyrene or polar-functionalized styrenes such as styrenes having hydroxyl, amino, nitrile, carboxylic acid, phosphonic acid, phosphoric acid, nitro or sulfonic acid groups and the salts thereof, in which the styrenes are polar-functionalized in the para position.
2) Unsaturated dicarboxylic or polycarboxylic acids, e.g. maleic acid esters such as dibutyl maleinate or dioctyl maleinate, as salts of allyl compounds, e.g. sodium allyl sulfonate, as salts of amyl derivatives, e.g. sodium amylsulfonate.
3) (Meth)acrylate comonomers, these are esters of acrylic acid and methacrylic acid, in which the ester groups are, for example, saturated or unsaturated, straight-chain, branched or cyclic hydrocarbon functional groups which can contain one or more heteroatoms, such as N, O, S, P, F, Cl, Br, I. Examples of such hydrocarbon functional groups are straight-chain, branched or cyclic alkyl, straight-chain, branched or cyclic alkenyl, aryl such as phenyl, or heterocyclyl such as tetrahydrofurfuryl.

Suitable (meth)acrylate comonomers, such as for AMPS, are for example:

a) acrylic acid, C₁-C₁₄ alkyl acrylic acid, such as methacrylic acid.
b) (meth)acrylamides, such as acrylamide, methacrylamide, diacetone-acrylamide, diacetone-methacrylamide, N-butoxymethyl-acrylamide, N-iso-butoxymethyl-acrylamide, N-butoxymethyl-methacrylamide, N-iso-butoxymethyl-methacrylamide, N-methylol acrylamide, N-methylol-methacrylamide.
c) heterocyclic (meth)acrylates, such as tetrahydrofurfuryl acrylate and tetrahydrofurfuryl methacrylate or carbocyclic (meth)acrylates, such as isobornyl acrylate and isobornyl methacrylate.
d) urethane (meth)acrylates, such as diurethane diacrylate and diurethane methacrylate (CAS: 72869-86-4).
e) C₁-C₁₄ alkyl acrylates such as methyl, ethyl, n-propyl, iso-propyl, n-butyl, sec. butyl, iso-butyl, tert. butyl, n-pentyl, iso-pentyl, hexyl (e.g. n-hexyl, iso-hexyl or cyclohexyl), heptyl, octyl (e.g. 2-ethylhexyl), nonyl, decyl (e.g. 2-propylheptyl or iso-decyl), undecyl, dodecyl, tridecyl (e.g. iso-tridecyl) and tetradecyl acrylate; the alkyl groups may optionally be substituted with one or more halogen atoms (e.g. fluorine, chlorine, bromine or iodine), e.g. trifluoroethyl acrylate, or with one or more amino groups, e.g. diethylaminoethyl acrylate, or with one or more alkoxy groups, such as methoxypropyl acrylate, or with one or more aryloxy groups such as phenoxyethyl acrylate.
f) C₂-C₁₄ alkenyl acrylates, such as ethenyl, n-propenyl, iso-propenyl, n-butenyl, sec. butenyl, iso-butenyl, tert. butenyl, n-pentenyl, iso-pentenyl, hexenyl (e.g. n-hexenyl, iso-hexenyl or cyclohexenyl), heptenyl, octenyl (e.g. 2-ethylhexenyl), nonenyl, decenyl (e.g. 2-propenylheptyl or iso-decenyl), undecenyl, dodecenyl, tridecenyl (e.g. iso-tridecenyl) and tetradecenyl acrylate, and the epoxides thereof, such as glycidyl acrylate or aziridines, such as aziridine acrylate.
g) C₁-C₁₄ hydroxyalkyl acrylates, such as hydroxymethyl, hydroxyethyl, hydroxy-n-propyl, hydroxy-iso-propyl, hydroxy-n-butyl, hydroxy-sec.-butyl, hydroxy-iso-butyl, hydroxy-tert.-butyl, hydroxy-n-pentyl, hydroxy-iso-pentyl, hydroxyhexyl (e.g. hydroxy-n-hexyl, hydroxy-iso-hexyl or hydroxy-cyclohexyl), hydroxyheptyl, hydroxyoctyl (e.g. 2-ethylhexyl), hydroxynonyl, hydroxydecyl (e.g. hydroxy-2-propylheptyl or hydroxy-iso-decyl), hydroxyundecyl, hydroxydodecyl, hydroxytridecyl (e.g. hydroxy-iso-tridecyl), and hydroxytetradecyl acrylate, wherein the hydroxyl group are in the terminal position (w position) (e.g. 4-hydroxy-n-butyl acrylate) or in the (ω-1)-position (e.g. 2-hydroxy-n-propyl acrylate) of the alkyl group.
h) alkylene glycol acrylates that contain one or more alkylene glycol units. Examples are i) monoalkylene glycol acrylates, such as acrylates of ethylene glycol, propylene glycol (e.g. 1,2- or 1,3-propanediol), butylene glycol (e.g. 1,2-, 1,3- or 1,4-butanediol, pentylene glycol (e.g. 1,5-pentanediol) or hexylene glycol (e.g. 1,6-hexanediol) in which the second hydroxyl group is etherified or esterified, e.g. by sulfuric acid, phosphoric acid, acrylic acid or methacrylic acid, or ii) polyalkylene glycol acrylates such as polyethylene glycol acrylates, polypropylene glycol acrylates, polybutylene glycol acrylates, polypentylene glycol acrylates or polyhexylene glycol acrylates, the second hydroxyl group of which may optionally be etherified or esterified, e.g. by sulfuric acid, phosphoric acid, acrylic acid or methacrylic acid.

Examples of (poly)alkylene glycol units having etherified hydroxyl groups are C₁-C₁₄ alkyloxy (poly)alkylene glycols (e.g. C₁-C₁₄ alkyloxy (poly)alkylene glycol acrylates), examples of (poly)alkylene glycol units having esterified hydroxyl groups are sulfonium (poly)alkylene glycols (e.g. sulfonium (poly)alkylene glycol acrylates) and the salts thereof, (poly)alkylene glycol diacrylates, such as 1,4-butanediol diacrylate or 1,6-hexanediol diacrylate or (poly)alkylene glycol methacrylate acrylates, such as 1,4-butanediol methacrylate acrylate or 1,6-hexanediol methacrylate acrylate;

The polyalkylene glycol acrylates may carry an acrylate group (e.g. polyethylene glycol monoacrylate, polypropylene glycol monoacrylate, polybutylene glycol monoacrylate, polypentylene glycol monoacrylate or polyhexylene glycol monoacrylate) or two or more, such as two, acrylate groups such as polyethylene glycol diacrylate, polypropylene glycol diacrylate, polybutylene glycol diacrylate, polypentylene glycol diacrylate or polyhexylenglycol diacrylate;

The polyalkylene glycol acrylates may also contain two or more different polyalkylene glycol blocks, e.g. blocks of polymethylene glycol and polyethylene glycol, or blocks of polyethylene glycol and polypropylene glycol;

The degree of polymerization of the polyalkylene glycol units or polyalkylene glycol blocks is generally in the range of from 1 to 20, such as in the range of from 3 to 10, i.e. in the range of from 3 to 6.

i) C₁-C₁₄ alkyl methacrylates such as methyl, ethyl, n-propyl, iso-propyl, n-butyl, sec. butyl, iso-butyl, tert. butyl, n-pentyl, iso-pentyl, hexyl (e.g. n-hexyl, iso-hexyl or cyclohexyl), heptyl, octyl (e.g. 2-ethylhexyl), nonyl, decyl (e.g. 2-propylheptyl or iso-decyl), undecyl, dodecyl, tridecyl (e.g. iso-tridecyl), and tetradecyl methacrylate; the alkyl groups may optionally be substituted with one or more halogen atoms (e.g. fluorine, chlorine, bromine or iodine), e.g. trifluoroethyl methacrylate, or with one or more amino groups, e.g. diethylaminoethyl methacrylate, or with one or more alkoxy groups, such as methoxypropyl methacrylate, or with one or more aryloxy groups, such as phenoxyethyl methacrylate.
j) C₂-C₁₄ alkenyl methacrylates, such as ethenyl, n-propenyl, iso-propenyl, n-butenyl, sec. butenyl, iso-butenyl, tert. butenyl, n-pentenyl, iso-pentenyl, hexenyl (e.g. n-hexenyl, iso-hexenyl or cyclohexenyl), heptenyl, octenyl (e.g. 2-ethylhexenyl), nonenyl, decenyl (e.g. 2-propenyl heptyl or iso-decenyl), undecenyl, dodecenyl, tridecenyl (e.g. iso-tridecenyl), and tetradecenyl methacrylate, and the epoxides thereof, such as glycidyl methacrylate or aziridines, such as aziridine methacrylate.
k) C₁-C₁₄ hydroxyalkyl methacrylates, such as hydroxymethyl, hydroxyethyl, hydroxy-n-propyl, hydroxy-iso-propyl, hydroxy-n-butyl, hydroxy-sec.-butyl, hydroxy-iso-butyl, hydroxy-tert.-butyl, hydroxy-n-pentyl, hydroxy-iso-pentyl, hydroxyhexyl (e.g. hydroxy-n-hexyl, hydroxy-iso-hexyl or hydroxy-cyclohexyl), hydroxyheptyl, hydroxyoctyl (e.g. 2-ethylhexyl), hydroxynonyl, hydroxydecyl (e.g. hydroxy-2-propylheptyl or hydroxy-iso-decyl), hydroxyundecyl, hydroxydodecyl, hydroxytridecyl (e.g. hydroxy-iso-tridecyl) and hydroxytetradecyl methacrylate, wherein the hydroxyl group is in the terminal position (ωposition) (e.g. 4-hydroxy-n-butyl methacrylate) or in the (ω-1)-position (e.g. 2-hydroxy-n-propyl methacrylate) of the alkyl group.
l) alkylene glycol methacrylates that contain one or more alkylene glycol units. Examples are i) monoalkylene glycol methacrylates, such as methacrylates of ethylene glycol, propylene glycol (e.g. 1,2- or 1,3-propanediol), butylene glycol (e.g. 1,2-, 1,3- or 1,4-butanediol), pentylene glycol (e.g. 1,5-pentanediol) or hexylene glycol (e.g. 1,6-hexanediol), in which the second hydroxyl group is etherified or esterified, e.g. by sulfuric acid, phosphoric acid, acrylic acid or methacrylic acid, or ii) polyalkylene glycol methacrylates, such as polyethylene glycol methacrylates, polypropylene glycol methacrylates, polybutylene glycol methacrylates, polypentylene glycol methacrylates or polyhexylene glycol methacrylates, the second hydroxyl group of which may optionally be etherified or esterified, e.g. by sulfuric acid, phosphoric acid, acrylic acid or methacrylic acid.

Examples of (poly)alkylene glycol units having etherified hydroxyl groups are C₁-C₁₄ alkyloxy (poly)alkylene glycols (e.g. C₁-C₁₄ alkyloxy (poly)alkylene glycol methacrylates), examples of (poly)alkylene glycol units having esterified hydroxyl groups are sulfonium (poly)alkylene glycols (e.g. sulfonium (poly)alkylene glycol methacrylates) and the salts thereof or (poly)alkylene glycol dimethacrylates, such as 1,4-butanediol dimethacrylate.

The polyalkylene glycol methacrylates can carry a methacrylate group (e.g. polyethylene glycol monomethacrylate, polypropylene glycol monomethacrylate, polybutylene glycol monomethacrylate, polypentylene glycol monomethacrylate or polyhexylene glycol monomethacrylate) or two or more, such as two, methacrylate groups, such as polyethylene glycol dimethacrylate, polypropylene glycol dimethacrylate, polybutylene glycol dimethacrylate, polypentylene glycol dimethacrylate or polyhexylene glycol dimethacrylate.

The polyalkylene glycol methacrylates may also contain two or more different polyalkylene glycol blocks, e.g. blocks of polymethylene glycol and polyethylene glycol or blocks of polyethylene glycol and polypropylene glycol (e.g. Bisomer PEM63PHD (Cognis), CAS 58916-75-9).

The degree of polymerization of the polyalkylene glycol units or polyalkylene glycol blocks is generally in the range of from 1 to 20, such as in the range of from 3 to 10, i.e. in the range of from 3 to 6.

Examples of non-limiting (meth)acrylate comonomers are 4-hydroxybutyl acrylate, 2-hydroxypropyl methacrylate, ammonium sulfatoethyl methacrylate, pentapropylene glycol methacrylate, acrylic acid, hexaethylene glycol methacrylate, hexapropylene glycol acrylate, hexaethylene glycol acrylate, hydroxyethyl methacrylate, polyalkylene glycol methacrylate (CAS No. 589-75-9), Bisomer PEM63PHD, methoxy polyethylene glycol methacrylate, 2-propylheptyl acrylate (2-PHA), 1,3-butanediol dimethacrylate (BDDMA), triethylene glycol dimethacrylate (TEGDMA), hydroxyethyl acrylate (HEA), 2-hydroxypropyl acrylate (HPA), ethylene glycol dimethacrylate (EGDMA), glycidyl methacrylate (GMA) and/or allyl methacrylate (ALMA).

The AMPS copolymers generally have a proportion of AMPS units of greater than 50 mol %, such as in the range of from 60-95 mol %, i.e. from 80 to 99 mol %, and the proportion of comonomers is generally less than 50 mol %, such as in the range of from 5 to 40 mol %, i.e. from 1 to 20 mol %.

The copolymers can be obtained by methods known per se, for example in the batch or in the semibatch method. For example, appropriate amounts of water and monomers are first conveyed into a temperature-controllable reactor and subjected to an inert gas atmosphere. The receiver is then stirred and brought to a reaction temperature (such as in the range of from approximately 70-80° C.) and an initiator is added, such as in the form of an aqueous solution. Suitable initiators are known initiators for radical polymerizations, for example sodium, potassium or ammonium peroxodisulfate, or H₂O₂-based mixtures, for example mixtures of H₂O₂ with citric acid. The maximum temperature is awaited, and as soon as the temperature in the reactor decreases either a) the remaining monomers are metered in, resulting in a post-reaction (semibatch method), or b) the post-reaction occurs directly (batch method). Thereafter, the reaction mixture obtained is cooled to room temperature and the copolymer is isolated from the aqueous solution, e.g. by extraction using organic solvents such as hexane or methylene chloride and subsequently distilling off the solvent. Thereafter, the copolymer may be washed and dried using organic solvent. The reaction mixture obtained can also be further processed directly, in which case it is advantageous to add a preservative to the aqueous copolymer solution.

The AMPS copolymers are suitable as protective colloids in the production of the inner microcapsules which can be used.

The production of the inner microcapsules, for example in the form of microcapsule dispersions, takes place by the at least one aromatic alcohol to be reacted and the at least one aldehydic component to be reacted, which has at least two C atoms per molecule, being brought together and reacted, optionally in the presence of at least one (meth)acrylate polymer, in the presence of the substance to be encapsulated (core material), i.e. the second fragrance composition, and the capsules are cured by means of subsequent increase in temperature. The pH may be increased during the course of the method.

Non-limitinq embodiments of a method may include
a) the at least one aromatic alcohol and/or the derivative or ether thereof and the at least one aldehydic component and optionally at least one (meth)acrylate polymer and at least one substance to be encapsulated are brought together at a temperature of from 40 to 65° C. and a pH between 6 and 9, such as between 7 and 8.5 and
b) the pH is raised to above 9, such as between 9.5 and 11, at a temperature of from 40 to 65° C. in a subsequent method step,
c) the capsules subsequently being cured by increasing the temperature to from 60° C. to 110° C., such as 70° C. to 90° C., in particular 80° C.

However, when phloroglucinol is used as the alcohol component, it is more advantageously cured in the acid range; the pH is then at most 4, such as between 3 and 4, for example between 3.2-3.5.

The inner capsules that can be produced are formaldehyde-free and can be easily processed as stable core/shell microcapsules from the aqueous slurry to form a dry flowable powder.

In further non-limitinq embodiments, the release from the inner microcapsules takes place by means of mechanical stress, in particular the inner microcapsules are friable. The inner microcapsules in such embodiments are substantially impermeable to the encapsulated fragrance composition, i.e. the diffusion of the encapsulated fragrance molecules is limited such that no or only a very small portion, usually below the perception threshold, of the odorant molecules penetrate the capsule shell by means of diffusion.

In embodiments, the capsule shell of the outer microcapsule is more permeable to the encapsulated first fragrance composition than the capsule shell of the inner microcapsule is to the encapsulated second fragrance composition. “More permeable” in this context means that the total amount of fragrance molecules which diffuses through the closed shell over a given period of time is greater than the reference. The amount is greater, for example, at least by a factor of 2, at least by a factor of 10, or at least by a factor of 100 or 1,000. As used herein, permeability or diffusion always refers to the capsules after use, i.e. for example after they have become attached to a surface, such as a textile. The diffusion therefore occurs into the ambient air, from a capsule which is already largely separated from the other constituents of the agent in which it was formulated.

In such embodiments, both the outer and inner microcapsules are broken up by means of mechanical stress and the contents are released, but the capsules differ in that the outer microcapsules are partially permeable to the fragrances encapsulated therein, i.e. at least more permeable than the inner microcapsules, so that the fragrances can be gradually released by means of diffusion, while the inner microcapsules are largely impermeable, i.e. less permeable to the fragrances encapsulated therein than the outer microcapsules, so that said fragrances are only released after the inner capsule shell has been broken up. However, the outer microcapsules do not allow the inner microcapsules to be released before the outer shell is broken up by means of mechanical stress. Since the outer microcapsules are broken up by mechanical stress, the inner microcapsules are released, which in turn are also broken up by means of mechanical stress. This results in the release of the first fragrance composition which has not already been released by means of diffusion, and at the same time the release of the second fragrance composition. The differences in the permeability between outer and inner microcapsules are for example as defined above and can be measured by means of TGA-FFIR.

The term “friable microcapsules” means microcapsules that can be opened or broken up by means of mechanical rubbing or by means of pressure, for example when drying one's hands with a towel, such that the contents are substantially only released as a result of mechanical action, for example when drying one's hands with a towel on which microcapsules of this kind are deposited. The used outer microcapsules based on melamine-formaldehyde resin and the inner microcapsules described herein are typically friable microcapsules of this kind.

In various embodiments, capsules for which permeability by diffusion is largely undesirable, such as the inner capsules, have >10 to 20% wall material, such as 12 to 18, alternatively 13-17, i.e. 14-16% wall material (=shell material) based on the total weight of the capsule, the remainder being formed by the core material. In conjunction with a conventional, suitable degree of cross-linking, this leads to a sufficient diffusion barrier of the inner capsule, for example. The inner capsules therefore, in various embodiments, have a proportion of from >10 to 20% wall material, such as 12 to 18, alternatively 13-17, i.e. 14-16% wall material relative to the total weight of the capsules.

In various other embodiments, a reduction of the wall material of the capsule of >10% leads to increased, faster and more perceptible diffusion of the core material. Accordingly, the outer capsules in various embodiments are characterized in that the proportion of the wall material relative to the total weight of the capsules is reduced by more than 10%, for example >10 to 30%, compared with the inner capsules. For example, the inner capsule may have a proportion of wall material of from 14-16%, in which case the outer capsule has a proportion of wall material of 13% or less, for example 10-12%.

The release behavior may also be regulated by the degree of cross-linking of the capsules, depending on the wall material (the terms “wall material” and “shell material” are used interchangeably herein). In addition to the reaction conditions (e.g. pH-value, time, temperature), this is also determined—in the case of melamine-formaldehyde capsules—by the molar ratio of formaldehyde to melamine.

It has been found in various embodiments that the molar ratio of formaldehyde to melamine can be adjusted in order to achieve the desired differences in the release behavior. These differences can be quantified by means of TGA-FFIR, as described above.

In various embodiments, these features are combined in terms of the amount of wall material and the degree of cross-linking, for example, by means of the ratio of formaldehyde-melamine in order to achieve the desired differences in release behavior.

In various embodiments, the outer microcapsule wall material comprises a polyacrylate, polyurethane, polyolefin, polyamide, polyester, polysaccharide, epoxy resin, silicone resin, and/or a polycondensation product consisting of carbonyl compounds and compounds containing NH groups. Melamine-urea-formaldehyde microcapsules or melamine-formaldehyde microcapsules or urea-formaldehyde microcapsules can be used as outer microcapsules, for example. In various embodiments, the capsule shell of the outer microcapsules consist substantially, i.e. by at least 50, such as at least 75, alternatively at least 90 wt. %, or completely of the aforementioned polymers, i.e. one thereof or a mixture of different polymers of the same substance class or different substance classes, in particular one thereof. The inner microcapsules are as described above, in particular those based on phloroglucinol and/or resorcinol, such as based on phloroglucinol, as component a). The capsule shell of the outer microcapsules may be substantially free of material which falls within the given definition of the material for the inner capsule shell. “Substantially free” means a content of less than 5 wt. %, such as less than 3 wt. %, for example less than 1 wt. %, in each case based on the total weight of the capsule shell, with no material of this kind (= below the detection limit).

It has surprisingly been found in the use of the capsule systems that the change in fragrance leads to a synergistic effect and the fragrance intensity is rated more highly. Furthermore, these capsule-in-capsule systems allow for a more distinct separation of the different fragrances, since the release of the fragrances from the inner capsule, in particular if this capsule is already substantially impermeable to the fragrances, is made more difficult by the encapsulation in an outer microcapsule. The odor profile of capsule-in-capsule systems of this kind thus differs from systems in which two different microcapsules have perfumes and capsule morphologies which correspond to the first and second fragrance compositions, respectively, (i.e. one capsule is diffusively permeable while the other is substantially impermeable), since using two separate capsules always leads to a certain degree of mixing of the individual fragrance compositions, whereas this mixing of the odor impressions is significantly reduced by the claimed capsule-in-capsule systems such that the different odors can be perceived as being more distinctly separated.

Another technical effect of the capsule systems is that although the inner capsules are high performance, they have the disadvantage that they exhibit undesirable discoloration and significant sedimentation when used in conventional agents. This disadvantage is due to the inner capsules being encapsulated in an outer capsule.

Non-limitinq microcapsules have average diameters (median of the size distribution) in the range of from 0.1 to 500 μm, such as between 1 and 150 μm, in particular between 1 and 100 μm, e.g. 10 to 80 μm. The shell of the microcapsules surrounding the core or (filled) hollow space has an average thickness in the range between advantageously approximately 1 nm and 1,000 nm, such as between approximately 10 nm and approximately 500 nm, for example between approximately 30 nm and approximately 300 nm, i.e. 30 nm to 200 nm, in particular approximately 50 nm to approximately 150 nm.

The outer microcapsules have average diameters (median of the size distribution) of from 5 to 500 μm, such as 10 to 150 μm, i.e. 15 to 100 μm, with shell thicknesses of from 30 nm to 200 nm, and the inner microcapsules have average diameters of from 1 to 30 μm, such as 2 to 25 μm, i.e. from 5 to 20 μm, with shell thicknesses of from 30 nm to 200 nm.

Among other things, the permeability of the shell, i.e. the diffusivity, can also be controlled by means of the shell thickness. Small shell thicknesses require a higher diffusive permeability than greater shell thicknesses. In various embodiments, the shell thickness of the outer microcapsule is approximately equal to or smaller than that of the inner microcapsule.

In embodiments, the outer microcapsule contains on average more than one, such as at least 2, for example at least 3, i.e. 4 or more inner microcapsules enclosed therein. In various embodiments, the outer microcapsule may contain up to 20, such as up to 15, i.e. up to 10, inner microcapsules.

In various embodiments, the average diameter of the inner capsules may be smaller than the average diameter of the outer capsules by at least a factor of 2, such as by a factor of 2-10, in particular 2-5. The outer microcapsules have for example an average diameter of from 20 to 80 μm, and the inner microcapsules have an average diameter of from 1 to 20 μm.

The inner microcapsules are dispersed in the first fragrance composition also encapsulated in the outer microcapsules. The capsule shell of both the outer and inner microcapsules is therefore insoluble in the first fragrance composition.

The first and second fragrance compositions each contain at least one fragrance. As fragrances or odorants or perfume oils, all known substances and mixtures can be used. The terms “odorant(s)”, “fragrances” and “perfume oil(s)” are used synonymously. This means in particular all those substances or the mixtures thereof which are perceived by humans and animals as an odor, in particular perceived by humans as a pleasant odor.

Perfumes, perfume oils or perfume oil components may be used as fragrance components. Perfume oils or fragrances may be individual odorant compounds, such as synthetic products of the ester, ether, aldehyde, ketone, alcohol and hydrocarbon types.

Fragrance compounds of the aldehyde type are, for example, adoxal (2,6,10-trimethyl-9-undecenal), anisaldehyde (4-methoxybenzaldehyde), cymal (3-(4-isopropyl-phenyl)-2-methylpropanal), ethylvanillin, Florhydral (3-(3-isopropylphenyl)butanal), helional (3-(3,4-methylenedioxyphenyl)-2-methylpropanal), heliotropin, hydroxycitronellal, lauraldehyde, Lyral (3- and 4-(4-hydroxy-4-methylpentyl)-3-cyclohexene-1-carboxaldehyde), methyl nonyl acetaldehyde, Lilial (3-(4-tert-butylphenyl)-2-methylpropanal), phenylacetaldehyde, undecylenealdehyde, vanillin, 2,6,10-trimethyl-9-undecenal, 3-dodecen-1-al, alpha-n-amylcinnamaldehyde, melonal (2,6-dimethyl-5-heptenal), 2,4-dimethyl-3-cyclohexene-1-carboxaldehyde (Triplal), 4-methoxybenzaldehyde, benzaldehyde, 3-(4-tert-butylphenyl)propanal, 2-methyl-3-(para-methoxyphenyl)propanal, 2-methyl-4-(2,6,6-timethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzylaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxybenzaldehyde, para-ethyl-alpha, alpha-dimethylhydrocinnamaldehyde, alpha-methyl-3,4-(methylenedioxy)hydrocinnamaldehyde, 3,4-methylenedioxybenzaldehyde, alpha-n-hexylcinnamaldehyde, m-cymene-7-carboxaldehyde, alpha-methylphenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexene carboxaldehyde, 1-dodecanal, 2,4-dimethylcyclohexene-3-carboxaldehyde, 4-(4-hydroxy-4-methylpentyl)-3-cyclohexene-1-carboxaldehyde, 7-methoxy-3,7-dimethyloctan-1-al, 2-methyl-undecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanindane-1- or 2-carboxaldehyde, 3,7-dimethyloctan-1-al, 1-undecanal, 10-undecen-1-al, 4-hydroxy-3-m ethoxybenzaldehyde, 1-methyl-3-(4-methylpentyl)-3-cyclohexenecarboxaldehyde, 7-hydroxy-3J-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexene-carboxaldehyde, 3J-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanindane-1-carboxaldehyde, 2-methyloctanal, alpha-methyl-4-(1-methylethyl)benzeneacetaldehyde, 6,6-dimethyl-2-norpinene-2-propionaldehyde, para-methylphenoxyacetaldehyde, 2-methyl-3-phenyl-2-propen-1-al, 3,5,5-trimethylhexanal, hexahydro-8,8-dimethyl-2-naphthaldehyde, 3-propyl-bicyclo-[2.2.1]-hept-5-en-2-carbaldehyde, 9-decenal, 3-methyl-5-phenyl-1-pentanal, methyl nonyl acetaldehyde, hexanal and trans-2-hexenal.

Fragrance compounds of the ketone type are, for example, methyl-beta-naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyl dihydrojasmonate, menthone, carvone, camphor, Koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, iso-E-Super (1-(1,2,3,4,5,6J,8-octahydro-2,3,8,8-tetramethyl-2-naphthalenyl)-ethan-1-one (and isomers)), methyl cedrenyl ketone, acetophenone, methyl acetophenone, para-methoxy acetophenone, methyl beta-naphthyl ketone, benzyl acetone, benzophenone, para-hydroxyphenyl butanone, celery ketone (3-methyl-5-propyl-2-cyclohexenone), 6-isopropyl decahydro-2-naphthone, dimethyloctenone, Frescomenthe (2-butan-2-yl-cyclohexan-1-one), 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, Dulcinyl(4-(1,3-benzodioxol-5-yl)butane-2-one), hexalone (1-(2,6,6-trimethyl-2-cyclohexene-1-yl)-1,6-heptadien-3-one), isocyclonE(2-acetonaphthone-1,2,3,4,5,6,7,8-octahydro-2,3,8,8-tetramethyl), methyl nonyl ketone, methyl cyclocitrone, methyl lavender ketone, orivone (4-tert-amylcyclohexanone), 4-tert-butylcyclohexanone, delphone (2-pentyl-cyclopentanone), muscone (CAS 541-91-3), neobutenone (1-(5,5-dimethyl-1-cyclohexenyl)pent-4-en-1-one), plicatone (CAS 41724-19-0), veloutone (2,2,5-trimethyl-5-pentylcyclopentan-1-one), 2,4,4,7-tetramethyl-oct-6-en-3-one and tetrameran (6,10-dimethylundecen-2-one).

Fragrance compounds of the alcohol type are, for example, 10-undecen-1-ol, 2,6-dimethylheptan-2-ol, 2-methylbutanol, 2-methylpentanol, 2-phenoxyethanol, 2-phenylpropanol, 2-tert-butycyclohexanol, 3,5,5-trimethylcyclohexanol, 3-hexanol, 3-methyl-5-phenylpentanol, 3-octanol, 3-phenyl-propanol, 4-heptenol, 4-isopropylcyclohexanol, 4-tert-butycyclohexanol, 6,8-dimethyl-2-nonanol, 6-nonen-1-ol, 9-decen-1-ol, α-methylbenzyl alcohol, α-terpineol, amyl salicylate, benzyl alcohol, benzyl salicylate, μ-terpineol, butyl salicylate, citronellol, cyclohexyl salicylate, decanol, dihydromyrcenol, dimethyl benzyl carbinol, dimethyl heptanol, dimethyl octanol, ethyl salicylate, ethyl vanillin, eugenol, farnesol, geraniol, heptanol, hexyl salicylate, isoborneol, isoeugenol, isopulegol, linalool, menthol, myrtenol, n-hexanol, nerol, nonanol, octanol, p-menthan-7-ol, phenylethyl alcohol, phenol, phenyl salicylate, tetrahydrogeraniol, tetrahydrolinalool, thymol, trans-2-cis-6-nonadicnol, trans-2-nonen-1-ol, trans-2-octenol, undecanol, vanillin, champiniol, hexenol and cinnamyl alcohol.

Fragrance compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, Floramat, Melusate, and Jasmacyclat.

Ethers include, for example, benzyl ethyl ether and Ambroxan. Hydrocarbons mainly include terpenes such as limonene and pinene.

Mixtures of different fragrances are used, which together produce an appealing fragrance note. Such a mixture of fragrances may also be referred to as perfume or perfume oil. Perfume oils of this kind may also contain natural fragrance mixtures, such as those obtainable from plant sources.

Fragrances of plant origin include essential oils such as angelica root oil, aniseed oil, arnica blossom oil, basil oil, bay oil, champaca blossom oil, citrus oil, abies alba oil, abies alba cone oil, elemi oil, eucalyptus oil, fennel oil, pine needle oil, galbanum oil, geranium oil, ginger grass oil, guaiac wood oil, gurjun balsam oil, helichrysum oil, ho oil, ginger oil, iris oil, jasmine oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, labdanum oil, lavender oil, lemon grass oil, lime blossom oil, lime oil, mandarin oil, melissa oil, mint oil, musk seed oil, muscatel oil, myrrh oil, clove oil, neroli oil, niaouli oil, olibanum oil, orange blossom oil, orange peel oil, oregano oil, palmarosa oil, patchouli oil, balsam Peru oil, petitgrain oil, pepper oil, peppermint oil, allspice oil, pine oil, rose oil, rosemary oil, sage oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil, and ambrettolide, Ambroxan, alpha-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornyl acetate, boisambrene forte, alpha-bromostyrene, n-decylaldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmine, camphor, carvacrol, carvone, p-cresol methyl ether, coumarin, p-methoxyacetophenone, methyl n-amyl ketone, methylanthranilic acid methyl ester, p-methylacetophenone, methyl chavicol, p-methylquinoline, methyl beta-naphthyl ketone, methyl n-nonyl acetaldehyde, methyl n-nonyl ketone, muscone, beta-naphthol ethyl ether, beta-naphthol methyl ether, nerol, n-nonylaldehyde, nonyl alcohol, n-octylaldehyde, p-oxyacetophenone, pentadecanolide, beta-phenethyl alcohol, phenylacetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, troenan, gamma-undecalactone, vanillin, veratraldehyde, cinnamaldehyde, cinnamyl alcohol, cinnamic acid, cinnamic acid ethyl ester, cinnamic acid benzyl ester, diphenyl oxide, limonene, linalool, linalyl acetate and propionate, melusate, menthol, menthone, methyl-n-heptenone, pinene, phenylacetaldehyde, terpinyl acetate, citral, citronellal and mixtures thereof.

In order to be perceptible, an odorant must be volatile, with the molar mass also playing an important role in addition to the nature of the functional groups and the structure of the chemical compound. Therefore, most odorants possess molar masses of up to approximately 200 daltons, whereas molar masses of 300 daltons and above represent something of an exception. Due to the differing volatility of odorants, the odor of a perfume or fragrance composed of multiple odorants varies over the course of vaporization, the odor impressions being divided into “top note”, “middle note” or “body” and “end note” or “dry out.” Analogously to the description in international patent publication WO 2016/200761 A2, the top, middle and end notes can be classified on the basis of their vapor pressure (determinable by means of the test methods described in WO 2016/200761) as follows:

Top note: vapor pressure at 25° C.: >0.0133 kPa

Middle note: vapor pressure at 25° C.: 0.0133 to 0.000133 kPa

End note: vapor pressure at 25° C.: <0.000133 kPa

Examples of tenacious odorants are essential oils such as angelica root oil, anise oil, arnica blossom oil, basil oil, bay oil, bergamot oil, champaca blossom oil, abies alba oil, abies alba cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemon grass oil, lime oil, mandarin oil, melissa oil, musk seed oil, myrrh oil, clove oil, neroli oil, niaouli oil, olibanum oil, orange oil, oregano oil, palmarosa oil, patchouli oil, balsam Peru oil, petitgrain oil, pepper oil, peppermint oil, allspice oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil, and cypress oil.

Higher-boiling or solid odorants of natural or synthetic origin include, for example: ambrettolide, α-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornyl acetate, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methylanthranilic acid methyl ester, p-methylacetophenone, methyl chavicol, p-methylquinoline, methyl-μ-naphthyl ketone, methyl n-nonyl acetaldehyde, methyl n-nonyl ketone, muscone, μ-naphthol ethyl ether, μ-naphthol methyl ether, nerol, nitrobenzene, n-nonyl aldehyde, nonyl alcohol, n-octylaldehyde, p-oxyacetophenone, pentadecanolide, μ-phenethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymene, thymol, γ-undecalactone, vanillin, veratraldehyde, cinnamaldehyde, cinnamyl alcohol, cinnamic acid, cinnamic acid ethyl ester, cinnamic acid benzyl ester

More volatile odorants include in particular lower-boiling odorants of natural or synthetic origin, which may be used alone or in mixtures. Examples of more volatile odorants are alkyl isothiocyanates (alkyl mustard oils), butanedione, limonene, linalool, linayl acetate and propionate, menthol, menthone, methyl-n-heptenone, phellandrene, phenylacetaldehyde, terpinyl acetate, citral, citronellal.

Non-limitinq fragrance compounds of the aldehyde type are hydroxycitronellal (CAS 107-75-5), helional (CAS 1205-17-0), citral (5392-40-5), bourgeonal (18127-01-0), Triplal (CAS 27939-60-2), ligustral (CAS 68039-48-5), vertocitral (CAS 68039-49-6), Florhydral (CAS 125109-85-5), citronellal (CAS 106-23-0), citronellyloxyacetaldehyde (CAS 7492-67-3).

In addition or as an alternative to the above-mentioned odorants, it is also possible to use the fragrances described in WO 2016/200761 A2, in particular the odorants mentioned in tables 1, 2 and 3, and the modulators listed in tables 4a and 4b. The publication in its entirety is incorporated herein by way of reference.

The microcapsules may also comprise other oils in addition to odorants. In particular, the microcapsules may also contain active substances in oil form, which are suitable for washing, cleaning, nourishing and/or finishing purposes, in particular
(a) textile care substances, such as silicone oils, and/or
(b) skin care substances, such as vitamin E, natural oils and/or cosmetic oils.

Skin care active substances are all those active substances which benefit the skin in a sensory and/or cosmetic manner. Skin care active substances are selected from the following substances:
a) waxes such as carnauba, spermaceti, beeswax, lanolin and/or derivatives thereof and others.
b) hydrophobic plant extracts
c) hydrocarbons such as squalene and/or squalane
d) higher fatty acids, such as those having at least 12 carbon atoms, for example lauric acid, stearic acid, behenic acid, myristic acid, palmitic acid, oleic acid, linoleic acid, linolenic acid, isostearic acid and/or polyunsaturated fatty acids and others.
e) higher fatty alcohols, such as those having at least 12 carbon atoms, for example, lauryl alcohol, cetyl alcohol, stearyl alcohol, oleyl alcohol, behenyl alcohol, cholesterol and/or 2-hexadecanol and others.
f) esters, such as cetyloctanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glyceryl distearate, glycerol tristearate, alkyl lactate, alkyl citrate and/or alkyl tartrate and others.
g) lipids such as cholesterol, ceramides and/or sucrose esters and others.
h) vitamins such as vitamins A, C and E, vitamin alkyl esters, including vitamin C alkyl esters and others.
i) sunscreens
j) phospholipids
k) derivatives of alpha-hydroxy acids
l) germicides for cosmetic use, both synthetic, such as salicylic acid and/or others, and natural, such as neem oil and/or others.
m) silicones
n) natural oils, e.g. almond oil and mixtures of any of the aforementioned components.

In various embodiments, the microcapsules additionally contain plant extracts as an active substance. These extracts are typically prepared by extraction of the entire plant. It may also be in some cases, however, to prepare the extracts solely from the flowers and/or leaves of the plant.

Extracts from green tea, oak bark, stinging nettle, witch hazel, hops, henna, chamomile, burdock root, horsetail, whitethorn, lime blossom, almond, aloe vera, pine needles, horse chestnut, sandalwood, juniper, coconut, mango, apricot, lime, wheat, kiwi, melon, orange, grapefruit, sage, rosemary, birch, hollyhock, cuckooflower, wild thyme, yarrow, thyme, melissa, rest harrow, coltsfoot, marshmallow, meristem, ginseng and ginger root are most suitable.

Water, alcohols and mixtures thereof can be used as extraction agents for preparing the mentioned plant extracts. Of the alcohols, low alcohols such as ethanol and isopropanol, but in particular polyhydric alcohols such as ethylene glycol and propylene glycol, are usable both as the sole extraction agent and in a mixture with water. Plant extracts based on water/propylene glycol in the ratio of 1:10 to 10:1 have proven to be particularly suitable.

The plant extracts can be used both in pure form and in diluted form. If they are used in diluted form, they typically contain approximately 2 to 80 wt. % of active substance and, as a solvent, the extraction agent or extraction agent mixture used for their extraction.

Furthermore, it may be optional to use a plurality of plant extracts, in particular two different plant extracts, as the active substance.

It may be advantageous that the outer/inner microcapsules which can be used can be particularly easily attached to the treated textile. This is achieved, for example, by the use of aminoplast capsules, such as those based on melamine-formaldehyde. After the washing process, in particular aminoplast capsules of this kind then usually have a particular brittleness, such that, by the action of mechanical force, a targeted release of active substance, in particular a release of fragrance, can take place from the capsule, e.g. when rubbing one's skin with a towel which has been washed with a corresponding washing agent. In this way, for example a pleasant odor can be induced in a targeted manner even after prolonged storage of the laundry. This pleasant odor that is induced in a targeted manner differs from the odor of the product, which also induced substantially by the conventional perfume or by the use of diffusively permeable outer microcapsules and the first fragrance composition, since said odor is dominated by the second fragrance composition released from the inner microcapsule, optionally in combination with the residues of the first fragrance composition which is released simultaneously from the outer microcapsules. This allows for a polysensory fragrance experience, i.e. an odor experience characteristic of the product as such and occurring when it is opened or applied, which odor experience is subsequently replaced by a later odor experience which occurs only after being applied. This enables the consumer to purposefully induce pleasant odors that differ from the odor of the washing agent.

In particular, the separation of the different fragrance compositions in different microcapsules offers the advantage of allowing for a comparatively clear separation of the fragrance perception of the different compositions and is far superior to known methods which are based on the mixture of fragrances of different volatilities.

The first and the second fragrance composition differ, for example, in terms of their fragrance profile and/or the volatility of the fragrances contained and/or the substantivity of the fragrances contained. The fragrance profile of the first and the second fragrance composition to differ for the consumer in terms of how they may be perceived by the senses. Fragrance profiles may be described, for example, as fresh, green, petrichor, floral, rose, lily of the valley, fruity, apple, berry, citrus, woody, cosmetic, balsamic, amber, musky and fougere, amongst others. Additionally or alternatively, the two fragrance compositions in the outer and inner capsules can also differ in the physicochemical composition, i.e. the fragrances/fragrance mixtures used differ in composition and physical parameters such as vapor pressure, boiling point, hydrophobicity (clogP value), etc.

In various embodiments, the first and the second fragrance composition differ in that the first fragrance composition contains at least one fragrance, such as two fragrances, for example three fragrances, i.e. four or more fragrances which are not contained in the second fragrance composition. Likewise, the second fragrance composition may contain at least one fragrance, such as two fragrances, for example three fragrances, i.e. four or more fragrances which are not contained in the first fragrance composition. However, this does not preclude both fragrance compositions from containing the same fragrances, as long as the compositions differ in at least one fragrance and/or the amounts of the fragrances used.

Generally, in various embodiments, the second fragrance composition, i.e. the composition in the inner capsule, may perform better than the first fragrance composition. This means that the odor profile of the second fragrance composition dominates even if the release is partially simultaneous, for example, if residues of the first are released together with the second fragrance composition by means of friction. Methods for achieving this, for example by means of selecting odorants on the basis of their vapor pressures, are known to a person skilled in the art.

The microcapsule systems described herein may contain the fragrance compositions in an amount of from 0.1 to 95 wt. %, such as 1 to 90 wt. %, i.e. 5 to 85 wt. %, based on the entire microcapsule system. The first fragrance composition constitutes at least 30, such as at least 50 wt. %, up to, for example, 80 wt. % or to 70 wt. % of the total amount of fragrance compositions in the capsule system. In various embodiments, the weight of the inner microcapsules constitutes up to 60 wt. %, such as 1 to 50 wt. %, for example 2 to 40 wt. % of the entire microcapsule system. In various embodiments, the weight of the polymer from which the outer capsule shell is made constitutes 1 to 25 wt. %, in particular 5 to 20 wt. %, of the total weight of the microcapsule system.

The microcapsule systems may be present in known forms, for example, as slurry in an aqueous carrier medium or as a powder.

Non-limitinq agents for washing, cleaning, conditioning, caring for and/or dyeing hard or soft surfaces contain microcapsules in amounts of from 0.0001 to 50 wt. %, such as 0.01 to 20 wt. %, and in particular 0.1 to 5 wt. %, based on the total agent.

The agents for washing, cleaning, conditioning, caring for and/or dyeing hard or soft surfaces in the context of this application are washing, cleaning, post-treatment and/or cosmetic agents.

The agents are used for washing, cleaning, conditioning, caring for and/or dyeing hard or soft surfaces. Hard surfaces in the context of this application are windows, mirrors and other glass surfaces, surfaces which consist of ceramic material, plastics material, metal or wood and lacquered wood, and have domestic and industrial uses, such as bathroom ceramics, cooking and dining utensils, kitchen surfaces or floors. Soft surfaces in the context of this application are textile fabrics, skin and hair.

Agents for washing hard or soft surfaces in the context of this application are textile-washing agents, for example formulations present in the form of powders, granules, beads, tablets, pastes, gels, wipes, bars or liquids.

Agents for cleaning hard or soft surfaces in the context of this application include all cleaners for hard or soft surfaces, in particular dishwashing detergents, all-purpose cleaners, toilet cleaners, sanitary cleaners and glass cleaners, toothpastes, skin cleaning agents, such as shower gels, or hair washing agents.

Agents for conditioning hard or soft surfaces in the context of this application are fabric softeners, fragrance rinsers, conditioning wipes for use in a tumble dryer, hygiene rinsers, deodorants, antiperspirants, hair-conditioning agents, styling agents and/or hair-setting agents.

Agents for caring for hard or soft surfaces in the context of this application are textile care agents, hair care agents or skin treatment agents, such as creams, lotions or gels.

Agents for dyeing hard or soft surfaces in the context of this application are hair-dyeing and hair-toning agents and agents for lightening keratin fibers.

In addition to the capsule-in-capsule systems described herein, the agents may additionally contain a conventional perfume. This perfume differs from both the first and the second fragrance composition of the systems, for example in terms of the parameters discussed above. This conventional perfume can give the product as such the actual odor which is perceived when opened or applied. In addition, the agents may also contain other conventional perfume microcapsules which contain the same or different perfumes.

The agents for washing, cleaning, conditioning, caring for and/or dyeing hard or soft surfaces allow odorants which are stored in the outer/inner capsules to be released in a targeted manner, but at the same time said agents have a separate odor which is typically determined by a conventional perfume of the product. The outer/inner capsules are stable within the agent-matrix and can be opened by a targeted stimulus, in particular mechanical force, resulting in the outer capsules additionally allowing the odorants to be released by means of diffusion. Mechanical force is understood to mean any type of force applied to the microcapsule, such as, for example, shearing forces, pressure and/or friction. During the application of the agent, for example when washing textiles or cleaning skin, the outer microcapsules are deposited on the hard or soft surface. The release of the fragrances from the outer microcapsules then takes place by diffusion, i.e. the fragrances migrate through the polymeric shell material and are thus slowly released. The microcapsules can then be easily opened, e.g. by means of friction, after the surface has dried, the friction also opening the inner microcapsules which were thus released from the outer microcapsules. In this way, the fragrance(s) of the residues of the first fragrance composition are released in a targeted manner from the outer capsules (the part that has not already been released by means of diffusion) and the second fragrance composition is released in a targeted manner from the inner capsules, such that the performance profile of the total agent is increased. In this process, in particular the fragrance effect is of particular importance since the product performance in many cases is judged by the consumer proportionally to the pleasant odor.

The agents for washing, cleaning, conditioning, caring for and/or dyeing hard or soft surfaces described herein allow a long-lasting release of fragrance, in particular long-lasting fragrancing and care of hard and soft surfaces (by the release from the outer microcapsule) and a targeted release of fragrance (by the release from the inner microcapsule and optionally also the outer microcapsule), even after long periods by using the microcapsule systems described herein.

In a non-limiting embodiment, the surface is a textile surface. If the surface is a textile surface, the agent for washing, cleaning, conditioning, caring for and/or dyeing hard or soft surfaces may be a washing, cleaning or post-treatment agent.

In a further embodiment, the surface is a body site, in particular skin and/or hair. If the surface is a body site, in particular skin and/or hair, the agent for washing, cleansing, conditioning, caring for and/or dyeing hard or soft surfaces may be a cosmetic composition.

The microcapsules can contact the skin and/or the hair either by means of direct contact of the skin and/or the hair with a cosmetic composition comprising microcapsules and/or by transferring the microcapsules by means of textiles which carry microcapsules of this kind on the surface.

In the methods also described herein for producing the microcapsule systems, first the inner microcapsules which contain the second fragrance composition are produced using methods known in the prior art. Said inner microcapsules are then mixed with the first fragrance composition, dispersed therein, and the resulting mixture is encapsulated using known methods, such that the outer microcapsules are formed.

The microcapsules that contain the second fragrance composition may be surface-modified in order to facilitate encapsulation in the outer microcapsule. For this purpose, the microcapsules that contain the second fragrance composition (inner microcapsules) are suspended in water, for example by means of one or more hydrophobic modifying agents. The modifying agents may be at least one compound selected from the group consisting of polyethyleneimides, quaternary ammonium compounds, such as those having hydrophobic hydrocarbon functional groups, quaternary polyvinylpyrrolidones and unsaturated fatty acids such as oleic acid. Examples of suitable quaternary ammonium compounds are betaine, choline chloride, benzalkonium chloride and di-(C₈₋₁₈ alkyl)dimethylammonium chloride, such as didecyldimethylammonium chloride. Suitable polyethyleneimide compounds are multifunctional ethyleneimide-based cationic polymers having molecular weights in the range of from 600 to 2,500,000 Da. Polymers of this kind are commercially available, for example, under the trade name Lupasol from BASF SE. Likewise, suitable quaternary PVPs are commercially available under the trade name Luviquat from BASF SE. In general, corresponding methods for modifying surfaces and encapsulating are described for example in EP 2 732 803 A1 for “disintegrants” and can be readily transferred to the inner microcapsules. After being modified, the microcapsules may be dried before then being combined with the first fragrance composition and encapsulated in the outer microcapsule.

It is clear that the surface-modified inner microcapsules described above in the context of the methods can also be used in the microcapsule systems. In various embodiments, the inner microcapsules are therefore surface-modified microcapsules as described above, in particular those which have been hydrophobically modified, such as those modified by at least one modifying agent selected from the group consisting of polyethyleneimides, quaternary ammonium compounds, quaternary polyvinylpyrrolidones and oleic acid.

By selecting and controlling the reaction conditions in the formation of the shells, for example controlling the shell thickness, the permeability thereof to the encapsulated odorants can be controlled. This can be used to produce, for example, inner microcapsule shells which are impermeable to the encapsulated odorants, and outer microcapsule shells which allow release by means of diffusion through the capsule shell.

Methods for producing polysensory fragrance impressions using the microcapsule systems described herein are also included. In these methods, the first fragrance composition is first released from the outer microcapsule and then, following a time delay, the second fragrance composition is released from the inner microcapsule. As a result, the fragrance profile can be modified and a polysensory fragrance impression for the consumer can be produced. The fragrance experience can be broadened by additionally using conventional, i.e. non-encapsulated, perfume in the agent. As a result, when opening and applying the product, there is a first fragrance impression substantially caused by the conventional perfume, and as the application cycle continues the first fragrance composition is released from the outer microcapsules by diffusion, and finally the second fragrance composition is released from the inner microcapsules under mechanical stress. In these methods, the agent containing the perfume and the microcapsules is brought into contact with a surface which the fragrances/microcapsules are deposited on/adsorbed by, and the encapsulated fragrances are then released following the appropriate stimulus.

In various embodiments of this method, the agent in which the capsule system described herein is used is a textile treatment agent, such as a washing agent or fabric softener, which additionally contains a conventional perfume. The capsule system used is based on melamine-formaldehyde-resin-based outer microcapsules and the inner microcapsules described herein based on aromatic alcohols, such as in particular phloroglucinol and/or resorcinol, such as phloroglucinol, the outer capsule being diffusely permeable and the inner capsule being closed, but both being friable. In a first step, before the product is applied, the top and middle notes of the conventional perfume are released and thus provide a first fragrance impression. After application, a moist textile is obtained, which is olfactorily characterized by the top and middle notes of the conventional perfume and by the fragrance composition diffusively released from the outer capsule. The dry textile is characterized by the middle and end notes of the conventional perfume and the fragrance composition diffusively released from the outer capsule and, after being subjected to mechanical stress, for example by means of friction or by the fabric being worn, the perfume exits the inner capsule and becomes dominant. It is clear that in the course of the application cycle, the odor profile of the conventional perfume quickly weakens and is gradually replaced by the odorants released from the outer microcapsule by means of diffusion. They are then replaced by the odorants from the inner microcapsule when mechanical stress is applied, i.e. when the dry textile is being worn or used.

In this way, the microcapsule systems described herein can be used to produce polysensory fragrance impressions.

The claimed agents for washing, cleaning, conditioning, caring for and/or dyeing hard or soft surfaces may, in addition to the microcapsules described, contain other ingredients, such as surface-active substances.

Possible surface-active substances are, in particular, anionic surfactants, non-ionic surfactants, cationic, zwitterionic, amphoteric surfactants and/or emulsifiers. However, the agent for washing, cleaning, conditioning, caring for and/or dyeing hard or soft surfaces may contain anionic, non-ionic and/or cationic surfactants. In particular, the use of a mixture of anionic and non-ionic surfactants is advantageous. The agent contains 0.05 wt. % to 50 wt. %, advantageously 1 to 40 wt. %, such as 3 to 30 wt. % and in particular 5 wt. % to 20 wt. % of a surface-active substance, in particular from the groups of anionic surfactants, non-ionic surfactants, cationic, zwitterionic, amphoteric surfactants and/or emulsifiers. This corresponds to a non-limitinq embodiment and allows for optimal cleaning performance.

The agent for washing, cleaning, conditioning, caring for and/or dyeing hard or soft surfaces may contain anionic surfactant, advantageously in amounts of from 0.1 to 25 wt. %, such as from 1 to 20 wt. %, in particular in amounts of from 3 to 15 wt. %, based on the total agent. This corresponds to a non-limitinq embodiment and allows for particularly advantageous cleaning performance. A particularly suitable anionic surfactant is alkylbenzene sulfonate, such as linear alkylbenzene sulfonate (LAS). A non-limitinq embodiment is achieved if the agent contains alkylbenzene sulfonate, advantageously in amounts of from 0.1 to 25 wt. %, more advantageously in amounts of from 1 to 20 wt. %, in particular in amounts of from 3 to 15 wt. %, based on the total agent.

Particularly suitable anionic surfactants are also the alkyl sulfates, in particular the fatty alcohol sulfates (FAS), such as C₁₂₋₁₈ fatty alcohol sulfate. C₈-C₁₈ alkyl sulfates can be used, such as C₁₃ alkyl sulfate and C₁₃₋₁₅ alkyl sulfate and C₁₃₋₁₇ alkyl sulfate, advantageously branched, in particular alkyl-branched, C₁₃₋₁₇ alkyl sulfate. Particularly suitable fatty alcohol sulfates are derived from lauryl and myristyl alcohol, i.e. fatty alcohol sulfates having 12 or 14 carbon atoms. The long-chain FAS types (C₁₆ to C₁₈) are very suitable for washing at higher temperatures. Alkyl sulfates which have a lower Krafft point, such as having a Krafft point of less than 45, 40, 30 or 20° C. may be used.

Krafft point is the term for the temperature at which the solubility of surfactants greatly increases due to the formation of micelles. The Krafft point is a triple point at which the solid or hydrated crystals of the surfactant are in equilibrium with the dissolved (hydrated) monomers and micelles thereof. The Krafft point is determined by a turbidity measurement according to DIN EN 13955: 2003-03. A non-limitinq embodiment is achieved if the agent for washing, cleaning, conditioning, caring for and/or dyeing hard or soft surfaces contains alkyl sulfate, in particular C₁₂-C₁₈ fatty alcohol sulfate, advantageously in amounts of from 0.1-25 wt. %, more advantageously 1-20 wt. %, in particular in amounts from 3-15 wt. %, based on the total agent.

Other non-limiting anionic surfactants include alkanesulfonates (e.g. secondary C13-C18 alkanesulfonate), methyl ester sulfonates (e.g. α-C12-C18 methyl ester sulfonate) and α-olefin sulfonates (e.g. α-C14-C18 olefinsulfonate) and alkyl ether sulfates (e.g. C12-C14 fatty alcohol-2EO-ether sulfate) and/or soaps. Other suitable anionic surfactants will be described below. However, FAS and/or LAS are particularly suitable.

The anionic surfactants, including the soaps, may be present in the form of the sodium, potassium or ammonium salts thereof or as soluble salts of organic bases, such as mono-, di- or triethanolamine. The anionic surfactants are present in the form of the sodium or potassium salts thereof, in particular in the form of the sodium salts.

The agent for washing, cleaning, conditioning, caring for and/or dyeing hard or soft surfaces may contain non-ionic surfactant, advantageously in amounts of from 0.01 to 25 wt. %, more advantageously from 1 to 20 wt. %, in particular in amounts of from 3 to 15 wt. %, based on the total agent. This corresponds to a non-limitinq embodiment. Alkylpolyglycol ethers may be used, in particular in combination with anionic surfactant, such as LAS.

Other suitable non-ionic surfactants are alkylphenol polyglycol ethers (APEO), (ethoxylated) sorbitan fatty acid esters (sorbitans), alkyl polyglucosides (APG), fatty acid glucamides, fatty acid ethoxylates, amine oxides, ethylene oxide-propylene oxide block polymers, polyglycerol fatty acid esters and/or fatty acid alkanolamides. Other suitable non-ionic surfactants will be described below. Sugar-based non-ionic surfactants, such as in particular APG may be used.

In another non-limiting embodiment, the surface-active substances are emulsifiers. Emulsifiers cause water-resistant or oil-resistant adsorption layers to form on the phase interface, which layers prevent dispersed droplets from coalescing and thus stabilize the emulsion. In a similar manner to surfactants, emulsifiers are therefore made up of a hydrophobic and a hydrophilic molecule part. Hydrophilic emulsifiers form O/W-emulsions and hydrophobic emulsifiers form W/O-emulsions. An emulsion is to be understood to mean a droplet-like distribution (dispersion) of a liquid in another liquid at the expense of energy to create stabilizing phase interfaces by means of surfactants. These emulsifying surfactants or emulsifiers are therefore to be selected according to the substances to be dispersed and the relevant external phase and particle size of the emulsion. Emulsifiers which may be used include
Addition products of 4 to 30 mol of ethylene oxide and/or 0 to 5 mol of propylene oxide to linear fatty alcohols having 8 to 22 C atoms, to fatty acids having 12 to 22 C atoms, and to alkyl phenols having 8 to 15 C atoms in the alkyl group,
C12-C22 fatty acid mono- and diesters of addition products of 1 to 30 mol of ethylene oxide to polyols having 3 to 6 carbon atoms, in particular to glycerol,
Ethylene oxide and polyglycerol addition products to methyl glucoside fatty acid esters, fatty acid alkanolamides and fatty acid glucamides,
C8-C22 alkyl mono- and oligoglycosides and the ethoxylated analogs thereof, with oligomerization degrees of from 1.1 to 5, in particular 1.2 to 2.0, and glucose as the sugar component,
Addition products of 5 to 60 mol ethylene oxide to castor oil and hydrogenated castor oil,
Partial esters of polyols having 3-6 carbon atoms with saturated fatty acids having 8 to 22 C atoms,
Sterols. Sterols are understood to refer to a group of steroids that carry a hydroxyl group at the C atom 3 of the steroid backbone and are isolated both from animal tissue (zoosterols) and from vegetable fats (phytosterols). Examples of zoosterols include cholesterol and lanosterol. Examples of suitable phytosterols include ergosterol, stigmasterol and cytosterol. There are also sterols that are isolated from fungi and yeasts (known as mycosterols).
Phospholipids. These are understood to mean principally the glucose phopholipids that are obtained from, for example, egg yolks or plant seeds (e.g. soy beans) as lecithins or phosphatidylcholines, for example.
Fatty acid esters of sugars and sugar alcohols, such as sorbitol,
Polyglycerols and polyglycerol derivatives, such as polyglycerol poly-12-hydroxystearate (commercial product: Dehymuls® PGPH),
Linear and branched fatty acids having 8 to 30 C atoms, and the Na, K, ammonium, Ca, Mg and Zn salts thereof.

When the agent for washing, cleaning, conditioning, caring for and/or dyeing hard or soft surfaces is a washing, cleaning or post-treatment agent, it may contain, in addition to the essential components, other ingredients which further improve the performance and/or aesthetic properties of the washing, cleaning or post-treatment agent. In the context, the washing, cleaning or post-treatment agent additionally contains one or more substances from the group of builders, bleaching agents, bleach catalysts, bleach activators, enzymes, electrolytes, non-aqueous solvents, pH adjusters, perfume compositions, perfume carriers, fluorescing agents, dyes, hydrotropes, suds suppressors, silicone oils, soil-release polymers, graying inhibitors, anti-shrink agents, anti-crease agents, dye transfer inhibitors, other antimicrobial active ingredients, germicides, fungicides, antioxidants, preservatives, corrosion inhibitors, antistatic agents, bittering agents, ironing aids, repellents and impregnating agents, swelling and anti-slip agents, softening components and UV absorbers.

Particular additional ingredients for washing, cleaning or post-treatment agents are builders, enzymes, electrolytes, non-aqueous solvents, pH adjusters, perfume compositions, fluorescing agents, dyes, hydrotropes, suds suppressors, soil-release polymers, graying inhibitors, dye transfer inhibitors, softening components, UV absorbers and mixtures thereof.

In a non-limiting embodiment, the washing, cleaning or post-treatment agents are in liquid form and contain water as the main solvent.

The invention also relates to the use of a washing, cleaning or post-treatment agent in the washing, cleaning and/or pre-treatment of textile fabrics.

When the agent for washing, cleaning, conditioning, caring for and/or dyeing hard or soft surfaces is a cosmetic composition, it may contain other ingredients in addition to the essential ingredients. The cosmetic composition also contains at least one cosmetic active ingredient from the group of oxidation dye precursors, direct dyes, oxidizing agents selected from hydrogen peroxide and the addition compounds thereof to solid carriers, hair-conditioning active ingredients, deodorizing and/or antiperspirant active ingredients, skin-lightening and/or skin-soothing and/or moisturizing active ingredients, inorganic and/or organic UV filter substances, sebum-regulating active ingredients, mechanical exfoliating agents, antimicrobial active ingredients, hair-setting or hairstyling active ingredients, anti-cariogenic active ingredients, calculus-prevention active ingredients and mixtures of these active ingredients. These cosmetic active ingredients are contained in an amount of from 0.01 to 70 wt. %, based on the total weight of the ready-to-apply agent.

The embodiments described in connection with the capsule systems according to the invention can likewise be transferred to methods for the production thereof, to the agents containing them and to the uses and methods described herein, and vice versa.

The invention is described below by means of examples, but is not limited thereto.

### EXAMPLES

### Example 1

### AMPS Hydroxybutyl Acrylate

891 g of demineralized water together with 585 g of AMPS (50% aqueous solution) and 7.5 g of 4-hydroxybutyl acrylate (HBA) were poured into the reactor and subjected to a protective-gas atmosphere. The reaction mixture was heated to 75° C. while stirring (400 rpm). 0.03 g of the water-soluble initiator sodium peroxodisulfate was dissolved in 15 g of water and injected into the reactor by means of a syringe when the reaction temperature was reached. After reaching the maximum temperature, one hour of post-reaction began. The batch was then cooled to room temperature and mixed with 1.5 g of preservative.

The aqueous solution was characterized by the viscosity, solids content and the pH. The viscosity was 540 mPas (measured at 20 rpm Brookfield), the solids content was 21% and the pH was 3.3. 3 g of copolymer was placed on a Petri dish and dried for 24 hours at 160° C. in a drying cabinet. The final weight is 0.69 g, which corresponds to a yield of 21.6%.

### Resorcinol Capsule

In a 400 ml beaker, 5.5 g of resorcinol was dissolved while stirring (stirring speed: approximately 1500 rpm) in 70 g of water and then mixed with 2.0 g of sodium carbonate solution (20 wt. %), whereupon the pH was approximately 7.9. This solution was heated to a temperature of approximately 52° C. Then, 25.5 g of glutardialdehyde was added.

The mixture was stirred for approximately a further 10 minutes at a stirring speed of approximately 1500 rpm and a temperature of approximately 52° C. (precondensation time). Thereafter, approximately 20 g of water was added and, approximately 2 minutes later, 1 g of the protective colloid AMPS hydroxybutyl acrylate (see above) was added and, approximately another 2 minutes later, the fragrance composition was added. Immediately thereafter, the stirring speed was increased to approximately 4,000 rpm and, at approximately the same time, 20.0 g of sodium carbonate solution (20 wt. %) was added. Thereafter, the pH of the mixture was approximately 9.7. In the subsequent period, the viscosity and the volume of the mixture increased. It was further stirred at a stirring speed of approximately 4,000 rpm until the viscosity dropped again. Only then was the stirring speed lowered to approximately 1,500 rpm. The batch was stirred approximately for a further 60 minutes at a temperature of approximately 52° C. and at approximately the same stirring speed. Thereafter, the mixture was heated to about 80° C. and the capsules were cured at this temperature over a period of 3 hours.

Capsule size distribution— D (90) 5-10 μm; encapsulation efficiency approximately 90%;

Drying yield>90%; solids of the slurry approximately 40 wt. %.

### Phloroglucinol Capsule

Analogously to the above method, the 5.5 g of resorcinol used therein was completely replaced by 6.3 g of phloroglucinol. Phloroglucinol microcapsules were thus obtained.

### Characterization of the Microcapsule Systems

A commercially available fabric softener (Vernel conc.) was mixed with a capsule system (sample 2; 0.3 wt. % of capsule slurry: inner phloroglucinol capsule is closed, friable and loaded with perfume 3, outer melamine-formaldehyde capsule is diffusive, friable and loaded with perfume 2) or a conventional capsule system (sample 1; 0.15 wt. % of phloroglucinol-capsule slurry: capsule is closed, friable and loaded with perfume 3) and the discoloration caused by the microcapsules and the sedimentation of the microcapsules were assessed by a panel of trained experts on a scale of 0-3 (0=no discoloration/sedimentation; 1=faint discoloration/sedimentation; 2=medium discoloration/sedimentation 3=severe discoloration/sedimentation).

### Assessment of Discoloration/Sedimentation of Phloroglucinol Capsules

| | Discoloration | Sedimentation |
|---|---|---|
| Sample 1 | 3 | 3 |
| Sample 2 | 0.9 | 1.2 |

It was found that the disadvantageous discoloration/sedimentation of the high-performance phloroglucinol capsules can be reduced considerably by being used in the capsule-in-capsule system.

The same experiment was carried out with resorcinol capsules, with the following results:

### Assessment of Discoloration/Sedimentation of Resorcinol Capsules

| | Discoloration | Sedimentation |
|---|---|---|
| Sample 1 | 2.1 | 2.3 |
| Sample 2 | 0.6 | 1.0 |

Here, an effect similar to that of the phloroglucinol capsule was observed.

1. A microcapsule system comprising an outer microcapsule having an outer capsule shell, wherein the outer microcapsule comprises:
at least one inner microcapsule enclosed therein having an inner capsule shell; and
a first fragrance composition;
wherein the outer capsule shell of the outer microcapsule completely surrounds the inner microcapsule and the first fragrance composition, wherein the inner microcapsule comprises a second fragrance composition completely surrounded by the inner capsule shell of the inner microcapsule and differs from the first fragrance composition; and
wherein the capsule shell of the inner microcapsule comprises a resin which can be obtained by reacting
i. at least one aromatic alcohol or the ether or derivatives thereof;
ii. at least one aldehydic component that comprises at least two C atoms per molecule; and
iii. optionally in the presence of at least one (meth)acrylate polymer.

2. The microcapsule system according to claim 1, wherein
the at least one aromatic alcohol is selected from the group consisting of phenols, o-cresol, m-cresol, p-cresol, α-naphthol, ρ-naphthol, thymol, pyrocatechol, resorcinol, hydroquinone, 1,4-naphthohydroquinone, phloroglucinol, pyrogallol, hydroxyhydroquinone, and mixtures thereof; and/or
the aldehydic component is selected from the group consisting of valeraldehyde, capronaldehyde, caprylaldehyde, decanal, succinic dialdehyde, cyclohexanecarbaldehyde, cyclopentanecarbaldehyde, 2-methyl-1-propanal, 2-methylpropionaldehyde, acetaldehyde, acrolein, aldosterone, antimycin A, 8′-apo-μ-caroten-8′-al, benzaldehyde, butanal, chloral, citral, citronellal, crotonaldehyde, dimethylaminobenzaldehyde, folinic acid, fosmidomycin, furfural, glutaraldehyde, glutardialdehyde, glyceraldehyde, glycolaldehyde, glyoxal, glyoxylic acid, heptanal, 2-hydroxybenzaldehyde, 3-hydroxybutanal, hydroxymethylfurfural, 4-hydroxynonenal, isobutanal, isobutyraldehyde, methacrolein, 2-methylundecanal, mucochloric acid, N-methylformamide, 2-nitrobenzaldehyde, nonanal, octanal, oleocanthal, orlistat, pentanal, phenylethanal, phycocyanin, piperonal, propanal, propenal, protocatechualdehyde, retinal, salicylaldehyde, secologanin, streptomycin, strophanthidin, tylosin, vanillin, cinnamaldehyde, and mixtures thereof.

3. The microcapsule system according to claim 1, wherein the at least one (meth)acrylate polymer is a copolymer of 2-acrylamido-2-methyl-propanesulfonic acid or the salts thereof with one or more additional (meth)acrylate monomers, wherein the one or more additional (meth)acrylate monomers are selected from the group consisting of acrylic acid, C₁₋₁₄ alkyl acrylic acid, (meth)acrylamides, heterocyclic (meth)acrylates, urethane (meth)acrylates, C₁₋₁₄ alkyl acrylates, C₂₋₁₄ alkenyl acrylates, C₁₋₁₄ hydroxyalkyl acrylates, alkylene glycol acrylates, C₁₋₁₄ alkyl methacrylates, C₂₋₁₄ alkenyl methacrylates, C₁₋₁₄ hydroxyalkyl methacrylates, alkylene glycol methacrylates, and combinations thereof.

4. The microcapsule system according to claim 1, wherein the outer capsule shell
comprises a resin selected from the group consisting of polyacrylates, polyurethanes, polyolefins, polyamides, polyesters, polysaccharides, epoxy resins, silicone resins, and/or polycondensation products; wherein the polycondensation products are selected from the group consisting of carbonyl compounds, compounds containing NH groups, and combinations thereof; and/or
is partially permeable at least to parts of the first fragrance composition; wherein the first fragrance compound is configured to be released by means of diffusion.

5. The microcapsule system according to claim 1, wherein the inner capsule shell is less permeable to the second fragrance composition than the outer capsule shell is to the first fragrance composition.

6. The microcapsule system according to claim 1, wherein the fragrance composition is configured to be released from the inner microcapsule and optionally also the outer microcapsule by means of mechanical stress.

7. The microcapsule system according to claim 1, wherein the first fragrance composition and the second fragrance composition differ with regard to their fragrance profile and/or the physicochemical properties.

8. A method for producing a microcapsule system according to claim 1, wherein the method comprises:
providing microcapsules comprising the second fragrance composition and a first fragrance composition;
encapsulating the microcapsules comprising the second fragrance composition and the first fragrance composition in an outer microcapsule.

9. An agent composition for washing, cleaning, conditioning, caring for and/or dyeing hard or soft surfaces, comprising the microcapsule system according to claim 1.

10. (canceled)
